(19) United States
(12) Patent Application Publication
(10) Pub. No.: US 2025/0072927 A1
(43) Pub. Date: Mar. 6, 2025

(54) TRANSECTION PATHWAYS FOR REDUCING PERICARDIAL RESTRAINT

(71) Applicant: EDWARDS LIFESCIENCES CORPORATION, Irvine, CA (US)

(72) Inventors: Allen Jeong Keel, Irvine, CA (US); Yaeer E. Lev, Torrance, CA (US); Atiya Makhdoom Ahmad, Irvine, CA (US); Zachary Christopher Berwick, Tustin, CA (US); Mazen Saadi Albaghdadi, Irvine, CA (US)

(21) Appl. No.: 18/955,704

(22) Filed: Nov. 21, 2024

Related U.S. Application Data
(63) Continuation of application No. PCT/US2023/023761, filed on May 26, 2023.
(60) Provisional application No. 63/347,514, filed on May 31, 2022.

(51) Int. Cl. A61B 17/32; A61B 17/00; A61B 18/00; A61B 18/14

(52) U.S. Cl. CPC A61B 17/320016; A61B 18/1445; A61B 18/1492; A61B 2017/00039; A61B 2018/00351; A61B 2018/146

(57) ABSTRACT

Methods of improving a heart function in a heart of a subject having heart dysfunction are described. An exemplary embodiment includes accessing a pericardial cavity of the heart and advancing a cutting device within the pericardial cavity to a first incision point. At least a parietal layer of a pericardium of the heart is then pierced. A cutting device is placed against the parietal layer and retracted along a selected cut path so as to create a first incision having a selected length in at least the parietal layer to reduce pressure exerted on the heart by the pericardium. The above operations are repeated to create one or more additional incisions along respective additional cut paths.

### RELATED APPLICATIONS

This application is a continuation application of International Patent Application No. PCT/US2023/023761, filed May 26, 2023, which claims the benefit of U.S. Provisional Application No. 63/347,514, filed on May 31, 2022. Each of the foregoing patent applications are hereby incorporated by reference in their entireties.

### TECHNICAL FIELD

This disclosure is directed to methods for treating heart failure, for example, heart failure with preserved ejection fraction (HFpEF) or reduced injection fraction (HFrEF) by providing transection pathways that comprise introducing one or more incision lengths in a pericardium, e.g., a pericardial layer, fibrous layer, and/or adipose tissue.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In an example, a method of improving a heart function in a heart of a subject having heart dysfunction is provided, the method comprising creating at least one incision length through a pericardium and reducing pressure exerted by the pericardium on a heart. In one example, the heart dysfunction is preserved ejection fraction or reduced injection fraction.

In one example, creating the incision length is through one or more of a parietal layer, a fibrous layer, and adipose tissue of the pericardium.

In another example, alone or in combination with any of the previous examples, the method further comprising, before creating the incision length, puncturing pericardial tissue and providing an access point into a pericardial space. In another example, alone or in combination with any of the previous examples, the method further comprises, before puncturing, providing subxiphoid access to the pericardium. In another example, alone or in combination with any of the previous examples, the method further comprises, before puncturing, providing transvascular access to the pericardium. The pericardium may be accessed, for instance, by sequentially accessing the jugular vein, the superior vena cava, the right atrium, the right atrial appendage (RAA), and the pericardial cavity.

In another example, alone or in combination with any of the previous examples, the method further comprises, after puncturing, inserting a guidewire into the pericardial space. In another example, alone or in combination with any of the previous examples, the method further comprises, advancing a dilator over the guidewire and into the pericardial space. In another example, alone or in combination with any of the previous examples, the method further comprises, after inserting a guidewire or dilator into the pericardial tissue, advancing a multi-lumen catheter over the guidewire into the pericardial space, the multi-lumen catheter having a proximal end, a distal end, and a longitudinal axis.

In another example, alone or in combination with any of the previous examples, the multi-lumen catheter comprises a pericardial transection device. In another example, alone or in combination with any of the previous examples, the multi-lumen catheter comprises a pericardial incision assembly. In another example, alone or in combination with any of the previous examples, the pericardial incision assembly comprises the pericardial transection device. In another example, alone or in combination with any of the previous examples, the pericardial transection device selected from a group consisting of a scalpel, a mechanical cutting device, an electrosurgical device, a reversibly retractable knife blade, an RF clamp, and combinations thereof.

In another example, alone or in combination with any of the previous examples, the device further comprises, a fiberscope and further obtaining visual information before, during, or after creating the at least one incision length through the pericardium. In another example, alone or in combination with any of the previous examples, the method further comprises advancing a sheath over the multi-lumen catheter, and into the pericardial space.

In another example, alone or in combination with any of the previous examples, the method further comprises, after advancing the multi-lumen catheter into the pericardial space, creating an opening at least through a parietal layer from within the pericardial space. In another example, alone or in combination with any of the previous examples, creating an opening through the parietal layer is performed using a scalpel, a mechanical cutting device, an electrosurgical device, a reversibly retractable knife blade, or a combination thereof.

In another example, alone or in combination with any of the previous examples, the method further comprises, after creating the opening in the parietal layer, introducing the scalpel, the mechanical cutting device, the electrosurgical device, or the reversibly retractable knife blade into the opening, wherein the scalpel, the mechanical cutting device, the electrosurgical device, or the reversibly retractable knife blade and reverse cutting the pericardium.

In another example, alone or in combination with any of the previous examples, the method further comprises, after advancing the multi-lumen catheter into the pericardial space, stabilizing a portion of the multi-lumen catheter within the pericardial space. In another example, alone or in combination with any of the previous examples, the stabilizing comprises deploying one or more stabilizing members, the one or more stabilizing members projecting laterally from the multi-lumen catheter. In another example, alone or in combination with any of the previous examples, the one or more stabilizing members are adjacent the distal end of multi-lumen catheter. In another example, alone or in combination with any of the previous examples, the one or more stabilizing members is selected from a wire, loop, or shape-memory metal. In another example, alone or in combination with any of the previous examples, the one or more stabilizing members is one or more inflatable structures.

In another example, alone or in combination with any of the previous examples, the method further comprises, after stabilizing the multi-lumen catheter into the pericardial space, securing a portion of the multi-lumen catheter within the pericardial space. In another example, alone or in combination with any of the previous examples, the method further comprises, after stabilizing the multi-lumen catheter in the pericardial space, creating an opening through the parietal layer from within the pericardial space using the scalpel, the mechanical cutting device, the electrosurgical device, or the reversibly retractable knife blade.

In another example, alone or in combination with any of the previous examples, the method further comprises, after stabilizing the multi-lumen catheter, securing a portion of the multi-lumen catheter in the pericardial space prior to or during either or both of creating an opening through the parietal layer and creating the incision length. In another example, alone or in combination with any of the previous examples, the method further comprises, introducing the scalpel, the mechanical cutting device, the electrosurgical device, or the reversibly retractable knife blade into the opening, the scalpel, the mechanical cutting device, the electrosurgical device, or the reversibly retractable knife blade arranged for reverse cutting.

The techniques, methods, operations, steps, etc. described or suggested in the above examples can be performed on a living subject (e.g., human, other animal, etc.) or on a simulation, (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc.

In another example, a method of improving a heart function in a heart of a subject having heart dysfunction on a simulation is provided, the method comprising: creating at least one incision length through a pericardium; and reducing pressure exerted by the pericardium on a heart.

In one aspect, the creating the incision length is through one or more of a parietal layer, a fibrous layer, and adipose tissue of the pericardium. In another example, alone or in combination with any of the previous examples, the simulated method further comprises, puncturing a pericardial space by inserting a guidewire into and advancing a dilator over the guidewire and into the pericardial space. In another aspect, alone or in combination with any of the previous aspects, the simulated method further comprises, before puncturing the pericardial space, providing subxiphoid access to the pericardium or transvascular access to the pericardium.

In another aspect, alone or in combination with any of the previous examples, the simulated method further comprises, after inserting a guidewire or dilator into the pericardial space, advancing a multi-lumen catheter over the guidewire into the pericardial space, the multi-lumen catheter having a proximal end, a distal end, a longitudinal axis, and comprising a pericardial transection device.

In another aspect, alone or in combination with any of the previous examples, the simulated method further comprises, creating the at least one incision length by cutting, perforating, clamping, gliding, scissoring, and/or shearing using the pericardial transection device.

In another aspect, alone or in combination with any of the previous examples, the pericardial transection device selected from a group consisting of a scalpel, a mechanical cutting device, an electrosurgical device, a reversibly retractable knife blade, and combinations thereof.

In another aspect, alone or in combination with any of the previous examples, the simulated method further comprises, reverse cutting the at least one incision length with the scalpel, the mechanical cutting device, the electrosurgical device, or the reversibly retractable knife blade. In another aspect, alone or in combination with any of the previous examples, the electrosurgical device comprises a cutting surface with one or more electrodes.

In another aspect, alone or in combination with any of the previous examples, the simulated method further comprises, a fiberscope, and further obtaining visual information before, during, or after creating the at least one incision length through the pericardium.

In another aspect, alone or in combination with any of the previous examples, the simulated method further comprises, after advancing the multi-lumen catheter into the pericardial space, stabilizing a portion of the multi-lumen catheter within the pericardial space, wherein the stabilizing comprises includes deploying one or more stabilizing members, the one or more stabilizing members projecting laterally from the multi-lumen catheter, for example, wherein the one or more stabilizing members is selected from a wire, loop, or shape-memory metal or is one or more inflatable structures

In another aspect, alone or in combination with any of the previous examples, creating the at least one incision length begins at an opening in the pericardial space and ends at the access point corresponding to either the subxiphoid access to the pericardium or the transvascular access to the pericardium.

In another aspect, alone or in combination with any of the previous examples, creating the at least one incision length comprises creating a plurality of incision lengths, for example, wherein the plurality of incision lengths are isolated from each other, or wherein at least one of the plurality of incision lengths intersect each other.

In another aspect, alone or in combination with any of the previous examples, the simulated method further comprises, ascertaining a location of a nerve, for example, at least a proximal location of at least a portion of a phrenic nerve using a nerve stimulating device or a nerve detecting device.

In another aspect, alone or in combination with any of the previous examples, the at least one incision length is chosen from: along a length or circumference of only the parietal layer of the pericardium; from an anterior to a posterior of a heart; from a posterior base to an apex of a heart; from a posterior right atrium to a apex of a heart; from a left ascending aorta to a apex of a heart; from a right ascending aorta to a apex of a heart; transversely about a heart; or combinations thereof.

In another example, a pericardial transection device is provided, the device comprising: a catheter comprising a distal end, at least one lumen, and a longitudinal axis; and an incision assembly coupled to the distal end of the catheter, wherein the incision assembly comprises a scalpel, a mechanical cutting device, an electrosurgical device, a reversibly retractable knife blade, or a combination thereof.

In one aspect, the catheter is a flexible multi-lumen catheter. In another aspect, alone or in combination with any of the previous examples, the electrosurgical device comprises a cutting surface with one or more electrodes.

In another aspect, alone or in combination with any of the previous examples, the device further comprises a sheath advanceable over the catheter. In another aspect, alone or in combination with any of the previous aspects, the total outer diameter (O.D.) is between about 6 Fr (2 mm) and about 30 Fr (10 mm), wherein the total outer diameter (O.D.) is between about 6 Fr (2 mm) and about 20 Fr (6.67 mm), wherein the catheter, including the sheath, has a total outer diameter (O.D.) of between about 6 Fr (2 mm) and about 15 Fr (5 mm), wherein the catheter, including the sheath, has a total outer diameter (O.D.) of between about 6 Fr (2 mm) and about 12 Fr (4 mm), wherein the catheter, including the sheath, has a total outer diameter (O.D.) of approximately 10 Fr (3.33 mm).

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1B of FIG. 1A depicting the serosal, visceral, fibrous layers and adipose tissue of the parietal pericardium, including the pericardial cavity.

FIG. 2A, 2B are anterior and posterior views, respectively depicting various exemplary incision paths in a pericardium by using a pericardial transection device in accordance with methods disclosed and described herein.

FIG. 3 is a depiction of an exemplary pericardial transection device for providing one or more incision paths in a pericardium, as disclosed and described herein.

FIG. 4 is a simplified diagram of a transvascular approach to the pericardial cavity, as disclosed and described herein.

FIG. 5 is a simplified diagram of an alternative transvascular approach to the pericardial cavity, as disclosed and described herein.

FIG. 6 is a simplified diagram of a parietal layer incision length and cut path as disclosed and described herein.

### DETAILED DESCRIPTION

The present disclosure provides for transection pathways for reducing pericardial restraint by incising or opening the pericardium with the intention of improving patient's health with heart dysfunction conditions. The present disclosure, in one example, provides cutting and/or incision paths of a subject's pericardium for relieving pericardial restraint and/or resolving a heart dysfunction. The present disclosure further provides for methods of treating heart dysfunction using the presently disclosed cut paths. In some examples, the cut paths and lengths are obtained using a medical device, such as a catheter, multi-lumen catheter, e.g., for transcatheter alleviation of pericardial restraint (TAPR).

The techniques, methods, operations, steps, etc. described or suggested herein or in the references incorporated herein can be performed on a living subject (e.g., human, other animal, etc.) or on a simulation, (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter or catheter, and the application of a directional force sufficient to cut or separate the tissue, the force being substantially in a direction towards the proximal end of the multi-lumen catheter or catheter, for example, by pulling the multi-lumen catheter or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an end point. An incision length can be linear, non-linear, or a plurality of linear and/or non-linear lengths that intersect or do not intersect about a curved or non-planar surface, such as the heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “heart dysfunction” is inclusive of the ordinary and customary meaning to one of ordinary skill in medical and surgical arts, for example, heart failure or congestive heart failure.

As used herein the phrase “pericardial transection device” is inclusive of a device with a cutting surface, for example an edge of a blade or a surface of an electrode.

As used herein the phrase “pericardial incision assembly” and “incision assembly” are used interchangeable and refer to an assemblage that includes a pericardial transection device.

As used herein the phrase “transcatheter device” is inclusive of a catheter structured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein, “delta pulmonary capillary wedge pressure, Δ(PCWP)” is the difference between pulmonary capillary wedge pressure (PCWP) measured on volume challenge (i.e. leg raise and/or fluid administration) and PCWP at rest.

As used herein the phrase “puncturing tip” is inclusive of an atraumatic object suitable for puncturing or penetrating tissue without substantial trauma to or bleeding from the vicinity of the picture or penetration.

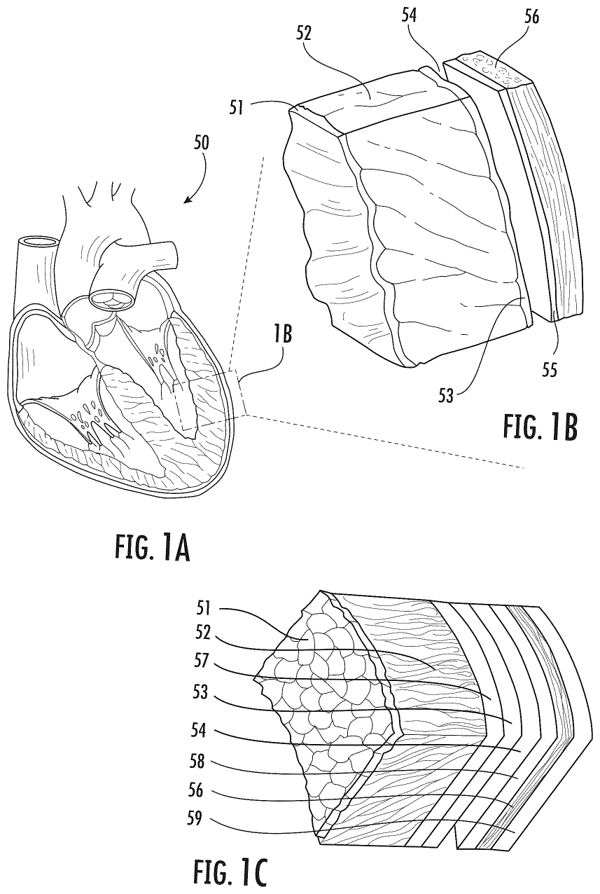

With reference to FIGS. 1A, 1B, 1C, and sections 1B, 1C. layers of a heart wall of a heart 50, from inside-out, being the endocardium 51, the myocardium 52, epicardial adipose tissue 57, the visceral layer of the serous pericardium 53, the pericardial cavity 54, the parietal layer 55 of the serous pericardium 58, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

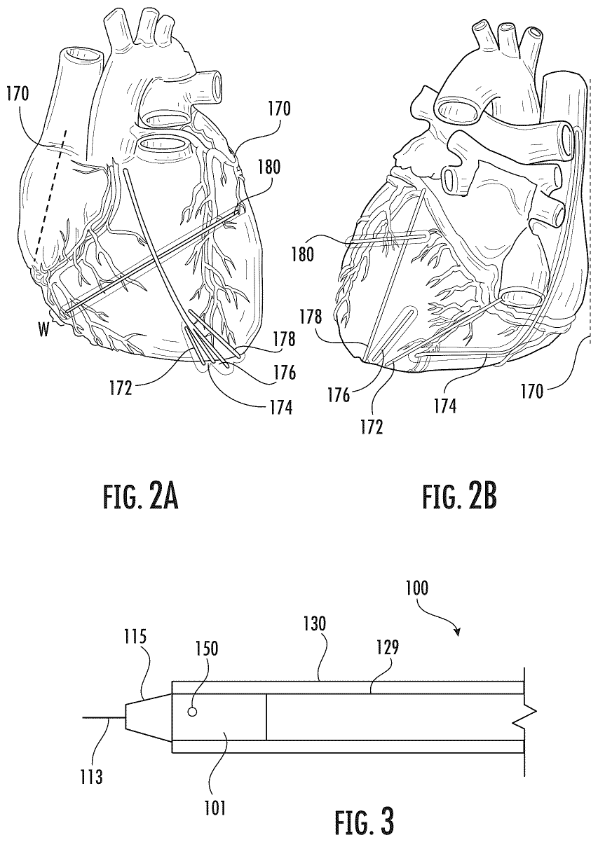

With reference to FIGS. 2A, 2B, at least one incision length is made in the pericardium. In some examples, an entry site is provided at the subxiphoid region of the patient. In some examples, fluoroscopy is used to visualize the position of the cannula and/or the pericardial transection device as it is advanced towards the heart. Visualization may be enhanced by incorporation of a radiopaque marker on one or more of the cannula and/or the pericardial transection device. In one example, the cannula is visualized from the left anterior oblique view at approximately 35-40°. In one example, the user may inject a contrast solution through the cannula or through a lumen of the pericardial transection device as they are being advanced towards the heart.

Part or all of the medical device (e.g., the multi-lumen catheter, the pericardial incision assembly, the pericardial transection device, the guidewire and controller) may be sterilized for use. Sterilization includes aseptic sterilization. The medical device may be sterilized using various sterilizing techniques, such as E-Beam sterilization, gamma sterilization, ethylene oxide sterilization, autoclave sterilization, chemical sterilization, and/or the like. Additionally, one or more materials used in the medical device may have anti-bacterial characteristics.

In one example, the cannula is advanced slowly towards the right ventricular apex of the heart, for example, to avoid vessels, etc. Once the pericardial transection device is positioned, a transection from a posterior side of the apex to an anterior base of a heart or in a reverse cutting device, posterior side of the apex to the anterior base of the heart. In one example, the at least one incision length is a path along a circumference of the pericardium of a length sufficient to reduce pericardial restraint. A sufficient length can be measured from an access point of the pericardial space to a distal location of the pericardial space from the access point, including an exit point in the pericardial space. In one example, the at least one incision length is measured as a percentage of the circumference of the heart being incised, for example at least 5, 10, 15, 20, 25, 30, 40, 50, 60, 70, 80, or 90 percent of the of the circumference of the heart being incised. In one example, the at least one incision length is more than 90 percent of the of the circumference of the heart being incised. In one example, a plurality of incision lengths are provided where each incision, or the total length of all of the plurality of incisions, are of a percent of the of the circumference of the heart being incised as stated above, so as to relieve pericardial restraint of the heart. The at least one incision can be planned and/or practiced preoperatively on a computerized and/or physical representation of a patient including simulated body parts. Such a simulation can include a cadaver, cadaver heart, virtual human, and/or imaginary person.

In one example, the at least one incision length is a path 172 made in the pericardium from an anterior apex to posterior base of a heart using a reverse cutting pericardial transection device (i.e. going over the middle cardiac vein and terminating the incision at the inferior vena cava). In one example, the at least one incision length is a path 174 from an apex of a heart to a posterior right atrium and terminating at the superior vena cava. In one example, the at least one incision length is a path 178 from an apex of a heart to a coronary sinus. In one example, the at least one incision length is a path 176 from an apex of a heart to an ascending aorta. In one example, the at least one incision length is a path 180 transversely about a heart (i.e. from a right ventricle to a left atrial appendage) about a heart. The incision lengths, in a heart with a dysfunction treatable with the present method, cause the pericardium to separate radially about the cut line as depicted schematically in the width W of the incision length of path 180 shown in FIG. 18, without the removal of pericardial tissue. Other incision lengths and paths may be employed, as well as combinations of incision lengths and paths, and combination of incision lengths and paths with one or more of partial removal of pericardium, drainage, and other pericardial treatments.

In one example, relief of pericardial restraint is at least 5 mmHg or more of the original pericardial restraint, as measured using delta PCWP. In some examples, relief of pericardial restraint is at least 5 mmHg. In some examples, relief of pericardial restraint is at least 10 mmHg. In some examples, relief of pericardial restraint is at least 15 mmHg. In some examples, relief of pericardial restraint is at least 20 mmHg. In some examples, relief of pericardial restrain is at least 25 mmHg. In some examples, relief of pericardial restraint is at least 30 mmHg. In some examples, relief of pericardial restraint is at least 35 mmHg. In some examples, relief of pericardial restraint is at least 40 mmHg. In some examples, relief of pericardial restraint is at least 45 mmHg. In some examples, relief of pericardial restrain is at least 50 mmHg. In some examples, relief of pericardial restraint is at least 55 mmHg. In some examples, relief of pericardial restrain is at least 60 mmHg. In some examples, relief of pericardial restraint is at least 65 mmHg. In some examples, relief of pericardial restraint is at least 70 mmHg. In some examples, relief of pericardial restraint is at least 75 mmHg. In some examples, relief of pericardial restraint is at least 70 mmHg. In some examples, relief of pericardial restraint is at least 65 mmHg. In some examples, relief of pericardial restraint is at least 60 mmHg. In some examples, relief of pericardial restraint is at least 55 mmHg. In some examples, relief of pericardial restraint is at least 50 mmHg. In some examples, relief of pericardial restraint is at least 45 mmHg. In some examples, relief of pericardial restraint is at least 40 mmHg. In some examples, relief of pericardial restraint is at least 35 mmHg. In some examples, relief of pericardial restraint is at least 30 mmHg. In some examples, relief of pericardial restraint is at least 25 mmHg. In some examples, relief of pericardial restraint is at least 20 mmHg. In some examples, relief of pericardial restraint is at least 15 mmHg. In some examples, relief of pericardial restraint is at least 10 mmHg. In some examples, relief of pericardial restraint is at least 5 mmHg.

In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method further comprises, after creating the at least one incision length, confirming a location of a distal end of the pericardial transection device; and in response to a signal indicative of a reduction of restraint of the heart, repeating the steps of creating the at least one incision length, and confirming a location of the distal end. In one example, a location of the distal end is confirmed by providing pairs or bars of radiopaque markers on opposite sides of the incision assembly 101 on the pericardium or within the pericardial cavity along the target cut path while the transection device is being advanced in the pericardial cavity to the starting point of the cut. As the one or more incisions lengths are created, a fluoroscope or other imaging technique can be used to visualize the separation of the pairs or bars of radiopaque markers, thereby indicating the length of the at least one incision.

In one example, the at least one incision length and path is preoperatively determined (e.g., on a simulation). In another example, creating at least one incision length is determined in response to a signal indicative of a reduction of restraint of the heart. In one example, creating at least one incision length is determined in response to a signal indicative of a reduction of restraint of the heart and repeating the creating of the at least one incision length in response thereto. In one example, the presently disclosed method further comprises, after creating the at least one incision length, confirming a location of a distal end of the pericardial transection device; and in response to a signal indicative of a reduction of restraint of the heart, repeating the steps of creating the at least one incision length, and confirming a location of the distal end.

In one example, the presently disclosed method further comprises, measuring a baseline parameter, and, after creating the at least one incision length, measuring an end hemodynamic parameter. In some examples, the entire length of the preoperatively determined target cut path is incised, before confirming the efficacy after the procedure with a right heart catheter pressure measurement and/or imaging. In some examples, another one or more incisions are created based upon a difference between the baseline parameter and the end parameter. For example, a measured pressure value from a pressure sensor or other hemodynamic parameter can be used to monitor a reduction in pericardial restraint or to target a threshold value to be reached and/or exceeded. In some examples, the one or more hemodynamic parameters or other parameters may be any of or any combination of the following: left arterial pressure, left ventricle volume, left ventricular contractility, intra-pericardial pressure, ventricular septum geometry, ventricular free wall geometry, size of particular portions of the heart, excursions of particular portions of the heart, electrical activity of the phrenic nerve 170, and the like. Additionally, or alternatively, the hemodynamic parameter is delta PCWP. In some such examples, the step of measuring an end hemodynamic parameter may be repeated to determine if an additional incision is desired.

In one example, an ultrasound method may be used to visualize the device and/or to measure the hemodynamic parameter. In some such examples, echocardiography is used. In some examples, a contrast dye is used. In yet another example, a fiber optic is used for visualization.

In another example, a bolus of saline is administered, and the measurement performed at operation is repeated while the saline effects are occurring. In some cases, the saline can be infused relatively rapidly. Before the effects of the saline bolus are complete, the measurement may be taken again. Next, pericardial transection is performed. The pericardial transection can be a single incision or multiple incisions. Optionally, another measurement, can be recorded after the effects of the saline bolus have substantially subsided. The value of the measured parameter may be repeated as many times as desired so long as the cut path is not completely incised. The values may be compared, to the baseline value, as well as to each other, to determine if additional cutting would yield more optimal results.

EXPERIMENTAL DESIGN: Using porcine hearts pressurized in the ventricles with pressurized water, the various pericardium cut paths depicted in FIGS. 2A, 2B were carried out and demonstrated various relief of pressures, efficacy, guidewire maneuverability, and safety considerations. The data generally showed relief (pressure drops) of varying degree for all cut paths and/or incision length along the paths. Thus, pericardial restraint relief can be provided using the methods disclosed and described herein by selecting a path length along the heart and creating a transverse incision of a length using a pericardial transection device.

The pericardial transection device can be any soft tissue cutting device, using a blade, current carrying wire, or combination thereof. The pericardial transection device can be a “scissor-like” instrument with more than one sharp edges. The pericardial transection device can be stabilized with one or more support members, such as wires or inflatable sacs or balloons. The support members can be passively or actively deployed during the procedure. The pericardial transection device can be structured to bulge, stretch or otherwise tension the pericardial space before or during incising.

Exemplary pericardial transection devices are not limited, and can include, for example, soft tissue cutting devices and the like. Exemplary pericardial transection devices can also include, for example, electrocautery devices.

With reference to FIG. 3, an exemplary pericardial transection device 100 is shown comprising a flexible catheter 129 comprising a distal end, at least one lumen, and a longitudinal axis; an incision assembly 101 coupled to the distal end of the catheter; and an introducer 115 coupled to and projecting from the incision assembly 101. In one example, the introducer 115 comprises a puncturing tip. In one example, at least a portion of the puncturing tip is radiopaque. In one example, at least a portion of the incision assembly 101 is radiopaque. In one example, at least a portion of the incision assembly 101 is radiopaque. The pericardial transection device and/or catheter and/or sheath can be structured such that the total outer diameter (O.D.) introduced to the pericardial cavity is between about 6 Fr (2 mm) and about 30 Fr (10 mm). The pericardial transection device and/or catheter and/or sheath can be structured such that the total outer diameter (O.D.) introduced to the pericardial cavity is between about 6 Fr (2 mm) and about 20 Fr (6.67 mm). The pericardial transection device and/or catheter and/or sheath can be structured such that the total outer diameter (O.D.) introduced to the pericardial cavity is between about 6 Fr (2 mm) and about 15 Fr (5 mm). The pericardial transection device and/or catheter and/or sheath can be structured such that the total outer diameter (O.D.) introduced to the pericardial cavity is between about 6 Fr (2 mm) and about 12 Fr (4 mm). The pericardial transection device and/or catheter and/or sheath can be structured such that the total outer diameter (O.D.) introduced to the pericardial cavity is approximately 10 Fr (3.33 mm).

In one example, the device 100 provides for creating elongated incisions of an incision length in at least the parietal layer, fibrous layer or adipose layer of the pericardium. The incision device comprises at least one cutting surface. The cutting surface can be a knife or scalpel or an electrode. In one example, the at least one incision length is a path along a circumference of the pericardium of a length sufficient to reduce pericardial restraint. The incision length can be made, either subxiphoidally, or transvascularly, by advancing an incision device, from the initial pericardial cavity entry location with cutting to a distal location (forward cutting). Alternatively, the incision length can be made by traversing the incision device from the initial pericardial cavity entry location to a distal location and then reversing back towards the entry location with cutting (reverse cutting). Thus, the presently disclosed method can be used with pericardial transection devices that are structured for either forward or reverse cutting modalities.

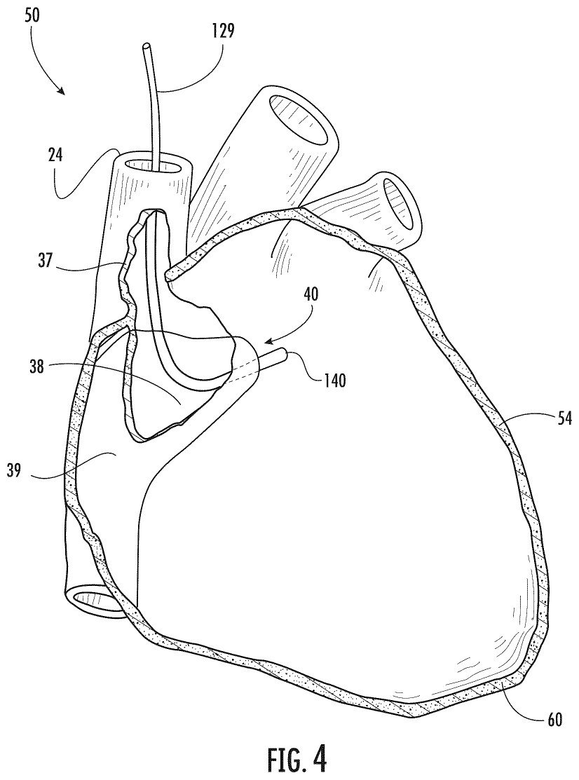

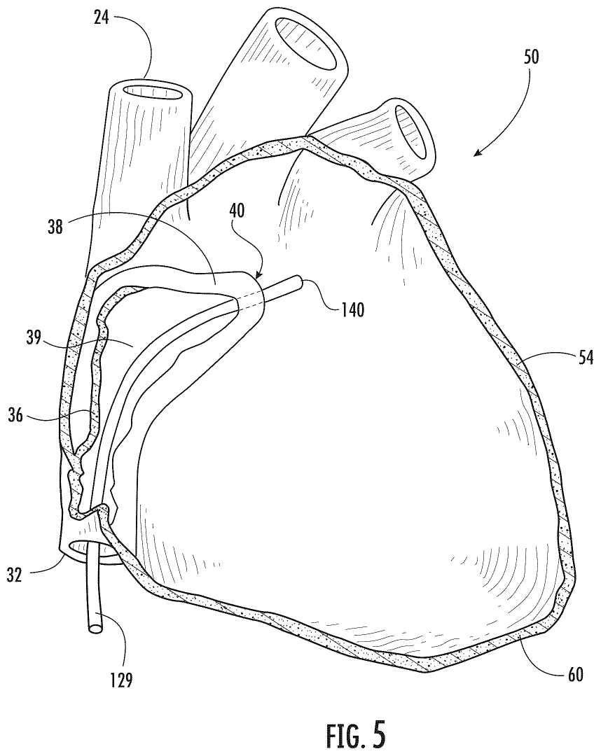

FIGS. 4-5 illustrate various intravascular approaches for delivering the transection devices to the pericardial cavity 54 and performing one or more cuts of one or more lengths. Thus, FIG. 4 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The exemplary transection device 100 may be presented to the pericardial cavity 54. In one example via the right atrial appendage 38 (RAA), a suitable site for entry into the pericardial cavity 54, is used. Right atrial appendage 38 lies tangential to and between pericardium 60 and the epicardium/epicardial adipose tissue 57. In one example, any of the presently disclosed transection devices 100 is guided into right atrial appendage 38 via right atrium 39 to be positioned substantially in parallel with the wall of pericardium 60 such that when the wall of right atrial appendage 38 is pierced by the transection device 100 it is done substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, the right atrial appendage 38 may be accessed via conventional vena cava routes. FIG. 4 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 5 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium appendage 38 at apex 40.

Thus, by way of example, the method of the present disclosure includes the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged.

The wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

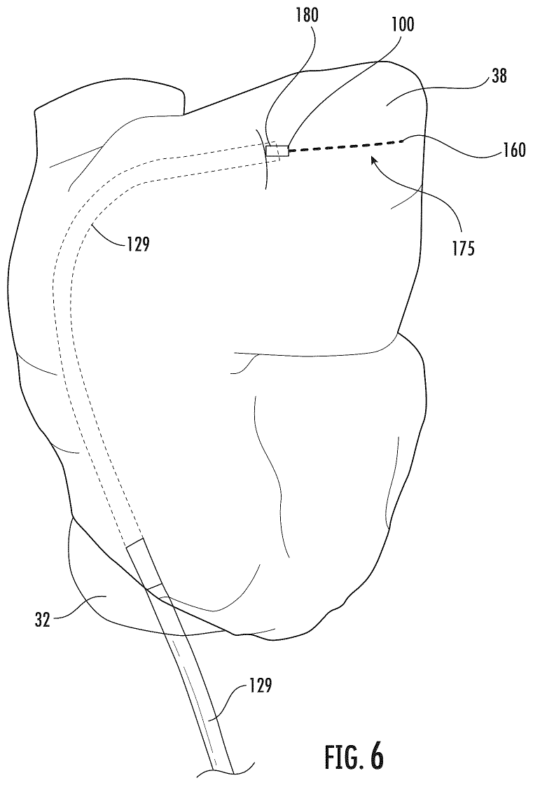

As shown in FIG. 6, a steerable catheter 129 may be employed, extending through the IVC, through the RA, and into the RAA and then into the pericardial cavity 54, the steerable catheter having a plurality of steerable segments. In some examples, the steerable catheter guiding the transection device 100 may have a radius of curvature of between about 1 inch and about 5 inches, with an arc length of between about 90° and about 180°. As shown in FIG. 6, transection device 100 begins a cut path 175 at a start point 160 and ends at endpoint 180. At least a portion of the parietal layer 55 of the serous pericardium 58, and the fibrous pericardium 56, and pericardial adipose tissue 59 are separated along cut path 175. The one or more incisions along the one or more lengths, in a heart with a dysfunction treatable with the present method, cause the pericardium to separate radially about the cut line of the cut path 175, without the removal of pericardial tissue. One or more cut paths 175 can be made, and different cut paths, of various lengths, e.g., segmented, continuous, or combinations, can be made in at least a portion of the parietal layer and other pericardium tissue to reduce pericardial restraint. In one example, the cut path 175 and its length is pre-operatively determined. Other cut paths and lengths can be used.

In one example, the device 100 can further comprise at least one nerve detection device. In one example, the at least one nerve detection device is located on the flexible catheter 129. In one example, the at least one nerve detection device is located adjacent the incision assembly 101. In one example, the at least one nerve detection device is located on the introducer/dilator 115. In one example, the at least one nerve detection device is located on the incision assembly 101.

In one example, a pacing catheter or pacing electrode can be used in combination with device 100. In another example, device 100 can be signal guided during deployment and/or during incising/cutting of the pericardium, for example, using impedance, echocardiography, or other methods.

In one example, the pericardial transection device 100 can further comprise at least one nerve proximity device 150. In one example, the at least one nerve proximity device 150 is a nerve detection device, for example, a pacing device or catheter introduced via a lumen in the transcatheter of the pericardial transection device. In one example, the at least one nerve detection device is located on a flexible catheter 129. In one example, the at least one nerve detection device is located adjacent the incision assembly 101. In one example, the at least one nerve detection device is located on the introducer/dilator 115. In one example, the at least one nerve detection device is located on the cutting surface. In one example, the at least one nerve detection device provides a proximity indication to at least a portion of a nerve, e.g., the phrenic nerve 170. The at least one nerve detection device can be an impedance sensor or other electrical field detection device that can provide a visual, audio and/or tactile indication to the user that the pericardial transection device is in proximity to at least a portion of a nerve.

In one example, the at least one nerve proximity device 150 is at least one nerve stimulation device. In one example, the at least one nerve stimulation device is located on the flexible catheter 129. In one example, the at least one nerve stimulation device is located adjacent the incision assembly 101. In one example, the at least one nerve stimulation device is located on introducer/dilator 115. In one example, the at least one nerve stimulation device is located on a cutting surface. In one example, the at least one nerve stimulation device provides a proximity indication to at least a portion of a nerve, e.g., the phrenic nerve 170. The at least one nerve stimulation device can be an electrical lead or other electrical discharging device that can provide a visual indication to the user that the pericardial transection device is in proximity to at least a portion of a nerve, e.g., a breathing change of the patient when in proximity to the phrenic nerve.

In one example, the presently disclosed device discussed above can further comprise an optical channel in the transcatheter to accommodate a lens coupled to a fiber optic cable, optionally with a light source, e.g., an LED. In one example, the presently disclosed method further comprises obtaining visual information during accessing, traversal of the pericardial cavity 54, exiting and/or cutting, for example, using an optical channel in the transcatheter to accommodate a lens coupled to a fiber optic cable, optionally with a light source, e.g., an LED.

A kit, comprising the pericardial transection device, a sheath 130, a guidewire 113, and a puncturing tip is provided.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

We claim:

1. A method of improving a heart function in a heart of a subject having heart dysfunction, the method comprising:
(a) accessing a pericardial cavity of the heart;
(b) advancing a cutting device within the pericardial cavity to a first incision point;
(c) piercing at least a parietal layer of a pericardium of the heart;
(d) retracting the cutting device along a selected cut path so as to create a first incision having a selected length in at least the parietal layer to reduce pressure exerted on the heart by the pericardium; and
(e) repeating operations (b) thru (d) to create one or more additional incisions along respective additional cut paths, wherein each of the first and the additional incisions are one of isolated from each other or intersect each other.

2. The method of claim 1, wherein creating the incision length is through one or more of the parietal layer, a fibrous layer, and adipose tissue of the pericardium.

3. The method of claim 1, wherein accessing the pericardial cavity comprises puncturing the pericardium to access the pericardial cavity and inserting a guidewire into and advancing a dilator over the guidewire and into the pericardial cavity.

4. The method of claim 3, further comprising, before puncturing the pericardium, providing subxiphoid access to the pericardium or transvascular access to the pericardium.

5. The method of claim 3, further comprising, after inserting a guidewire or dilator into the pericardial cavity, advancing a multi-lumen catheter over the guidewire into the pericardial cavity, the multi-lumen catheter having a proximal end, a distal end, a longitudinal axis, and comprising the cutting device.

6. The method of claim 5, wherein retracting the cutting device comprises pulling the cutting device along the guidewire to create the first incision and the additional incisions.

7. The method of claim 1, wherein the cutting device selected from a group consisting of a scalpel, a mechanical cutting device, an electrosurgical device, a reversibly retractable knife blade, and combinations thereof.

8. The method of claim 1, wherein the cutting device comprises first and second cutting surfaces in a scissor-shaped configuration, wherein piercing at least the parietal layer further comprises placing one cutting surface above the parietal layer and the second cutting surface below the parietal layer, and wherein retracting the cutting device comprises retracting the cutting device such that the first and second cutting surfaces cut through the parietal layer.

9. The method of claim 8, wherein the first cutting surface is an electrode and the second cutting surface is a mechanical blade, and wherein retracting the cutting device further comprises applying power to the electrode of the first cutting surface during retraction.

10. The method of claim 9, wherein the first cutting surface comprises two or more electrodes.

11. The method of claim 5, wherein the multi-lumen catheter further comprises a fiberscope and the method further comprises obtaining visual information before, during, or after creating the at least one incision length through the pericardium.

12. The method of claim 5, further comprising, after advancing the multi-lumen catheter into the pericardial space, stabilizing a portion of the multi-lumen catheter within the pericardial cavity, wherein the stabilizing comprises deploying one or more stabilizing members, the one or more stabilizing members projecting laterally from the multi-lumen catheter.

13. The method of claim 1, further comprising detecting a phrenic nerve.

14. The method of claim 13, wherein said retracting the cutting device along a selected cut path so as to create a first incision having a selected length in at least the parietal layer is selected so as to avoid cutting the phrenic nerve.

15. The method of claim 1, wherein the at least one incision length is chosen from: along a length or circumference of only the parietal layer of the pericardium; from an anterior to a posterior of a heart; from a posterior base to an apex of a heart; from a posterior right atrium to an apex of a heart; from a left ascending aorta to an apex of a heart; from a right ascending aorta to an apex of a heart; or transversely about a heart.

16. A method of improving a heart function in a heart of a subject having heart dysfunction, the method comprising:
(a) accessing a pericardial cavity of the heart;
(b) advancing a cutting device within the pericardial cavity to a first incision point, wherein the cutting device comprises first and second cutting surfaces in a scissor-shaped configuration;
(c) piercing at least a parietal layer of a pericardium of the heart;
(d) placing one cutting surface of the cutting device above the parietal layer and the second cutting surface below the parietal layer;
(e) retracting the cutting device along a selected cut path such that the first and second cutting surfaces cut through the parietal layer and so as to create a first incision having a selected length in at least the parietal layer to reduce pressure exerted on the heart by the pericardium; and
(f) repeating operations (b) thru (e) to create one or more additional incisions along respective additional cut paths, wherein each of the first and the additional incisions are one of isolated from each other or intersect each other.

17. The method of claim 16, wherein the first cutting surface is an electrode and the second cutting surface is a mechanical blade, and wherein retracting the cutting device further comprises applying power to the electrode of the first cutting surface during retraction.

18. The method of claim 16 further comprising stabilizing the cutting device within the pericardial cavity by deploying one or more stabilizing members into the pericardial cavity.

19. The method of claim 16, wherein the at least one incision length is chosen from: along a length or circumference of only the parietal layer of the pericardium; from an anterior to a posterior of a heart; from a posterior base to an apex of a heart; from a posterior right atrium to an apex of a heart; from a left ascending aorta to an apex of a heart; from a right ascending aorta to an apex of a heart; or transversely about a heart.

20. The method of claim 16, further comprising detecting a phrenic nerve and wherein said retracting the cutting device along a selected cut path so as to create a first incision having a selected length in at least the parietal layer is selected so as to avoid cutting the phrenic nerve.
